(19) United States
(12) Patent Application Publication
(10) Pub. No.: US 2026/0283146 A1
(43) Pub. Date: Sep. 24, 2026

(54) AFTERLOAD DEVICE FOR HEART EVALUATION

(71) Applicant: LUND HEART EVALUATION AB, Genarp (SE)

(72) Inventors: Henry PIGOT, Lund (SE); Tomas NORLANDER, Höör (SE); Kristian SOLTESZ, Lund (SE)

(73) Assignee: LUND HEART EVALUATION AB, Genarp (SE)

(21) Appl. No.: 19/484,743

(22) PCT Filed: May 17, 2024
(86) PCT No.: PCT/SE2024/050479

§ 371 Date: Nov. 14, 2025

(30) Foreign Application Priority Data
May 17, 2023 (SE) 2330226-8

(51) Int. Cl. A01N 1/143; A01N 1/165

(52) U.S. Cl. CPC A01N 1/143; A01N 1/165

(57) ABSTRACT

Method and device for providing an afterload to a beating heart. The afterload device is connected to the heart for receiving fluid flow expelled by the ventricle and comprises an afterload body, a valve body and a positioning device for positioning the valve body in the afterload body for forming an adjustable resistor. A control device receives input data, such as pressure and/or fluid flow rates and/or valve body position, and is arranged to adjust the adjustable resistor to have a first conductance at the start of the systolic phase, which increases during a first part of the systolic phase followed by a decrease of conductance during a second part of the systolic phase. An auxiliary fluid flow is provided to the connection between the afterload device and the ventricle, which is divided in a reverse fluid flow towards the ventricle and a fluid flow through the resistor.

### FIELD OF INVENTION

The present invention relates to a device and method for evaluating a heart intended for transplantation. In particular, the device is arranged to expose the beating heart ex vivo for a dynamic afterload comprising resistance, compliance and inertance that are dynamically adjustable. The device and method may also be used in scientific research of the heart, for example to study the influence of different drugs on the heart or testing different operating conditions.

### BACKGROUND ART

In an article published 1912 with the title: “The influence of variations in temperature and blood-pressure on the performance of the isolated mammalian heart”, by F. P. Knowlton and E. H. Starling, from the Institute of Physiology, University College, London, there is disclosed a method by which it is possible to determine the output of the left ventricle under approximately normal conditions, and to vary at will the arterial resistance, the venous pressure, the filling of the heart, or the temperature of the blood supply to the heart. This device is the basis for many later developed afterload devices for determining the output of the heart.

In another article with the title: “The arterial Windkessel” by Nico Westerhof, Jan-Willem Lankhaar and Berend E. Westerhof, published 2009, a lumped model of a so-called Windkessel model is disclosed. Another article with title: “A controllable artificial afterload for isolated heart studies”, by A. Fisher, R. E. Challis and P. Swann describes a similar system.

Patent document U.S. Pat. No. 7,045,279 discloses an isolated heart preparation in which essentially normal pumping activity of all four chambers of the heart is preserved, allowing for the use of the preparation in conjunction with investigations of electrode leads, catheters, cardiac implants and other medical devices intended to be used in or on a beating heart. The preparation may also be employed to investigate heart functions, in the presence or absence of such medical devices. In order to allow for visualization of heart structures and devices located within the chambers of the heart, a clear perfusate such as a modified Krebs buffer solution with oxygenation is circulated through all four chambers of the heart and the coronary vasculature. The preparation and recordings of the preparation may be used in conjunction with the design, development, and evaluation of devices for use in or on the heart, as well as for use as an investigational and teaching aid to assist physicians and students in understanding the operation of the heart.

U.S. Pat. No. 10,405,756B2 discloses an examination device for a heart. An afterload device connected to the aorta provides a counterpressure by an annular space delimited between a rigid cylinder and an elastic tube inserted in the rigid cylinder. The annular space comprises a compressible medium, such as air, nitrogen gas or carbon dioxide and is connected to a reservoir providing a predetermined pressure corresponding to a diastolic pressure. The reservoir is connected to the annular space via a restriction and a back-flow valve. During diastole, the annular space is inflated by the reservoir and provides a diastolic counter pressure for providing coronary flow. During systole, the ejected fluid from the heart ventricle displaces the medium inside the annular space through the restriction to the reservoir, thereby removing energy from the fluid. The compressible medium forms a compliance. In addition, there is a preload device comprising a vertical collapsible tube forming a fluid column for preload of the atrium with a desired preload pressure without introducing air into the fluid. A pump delivers fluid to the preload device and removes fluid from the afterload device. However, it is difficult to adjust compliance and inertance in this device.

There is a need for a more versatile afterload device, which may dynamically simulate the conditions of a heart and a body in vivo and expose the heart ex vivo to different afterloads.

### SUMMARY OF THE INVENTION

Accordingly, an object of the present invention is to mitigate, alleviate or eliminate one or more of the above-identified and other deficiencies and disadvantages singly or in any combination.

In an aspect, there is provided a method of providing an afterload to a beating heart ex vivo, comprising providing a first fluid flow to an atrium of the heart, whereupon the heart expels the provided fluid flow by means of a ventricle during a single heart beat in an outflow flow during a systolic phase followed by a diastolic phase in which there is no outflow flow from the ventricle; passing the outflow flow to an afterload device comprising an adjustable first resistor connected to the ventricle of the heart; providing fluid flow to coronary vessels of the heart; adjusting the first resistor to have at the start of the systolic phase a first conductance, which increases during a first part of the systolic phase and decreases during a following second part of the systolic phase during said single heart beat.

In an embodiment, the method further comprises providing a second fluid flow to the connection between the afterload device and the ventricle, whereby the second fluid flow, during the diastolic phase, is divided into a third fluid flow towards the ventricle and a fourth fluid flow through the first resistor. The second fluid flow may be provided during both the systolic and the diastolic phases. Alternatively, the second fluid flow is provided only during the diastolic phase, and optionally also during at least part of the systolic phase. If provided during the systolic phase, the third fluid flow is directed through the first resistor.

In an embodiment, in which the left portion of the heart is operated, the second fluid flow is provided to the connection between the afterload device and a left ventricle of the heart, whereupon the third fluid flows towards the left ventricle during the diastolic phase, thereby closing a corresponding aortic valve and providing said third fluid flow to the coronary vessels of the heart. The second second flow may be provided with a predetermined second fluid flow rate, which is larger than a required fluid flow to the coronary vessels, and wherein the resistor is adjusted so that the fourth fluid flow through the first resistor causes a pressure drop over the first resistor, which is equal to a predetermined diastolic pressure. The predetermined diastolic pressure may comprise a start-diastolic pressure which decreases during the diastolic phase, or alternatively is constant. Alternatively, the second fluid flow may be provided by a constant pressure pump providing a constant pressure which is larger than the predetermined diastolic pressure and is provided to the connection between the afterload device and a left ventricle of the heart via a second resistor, whereby the first resistor during the diastolic phase is adjusted so that the provided constant pressure is divided to produce said predetermined diastolic pressure at said connection.

In another embodiment, in which the right portion of the heart is operated, the second fluid flow may be provided to the connection between the afterload device and a right ventricle of the heart, whereupon the third fluid flow towards the right ventricle closes a corresponding pulmonary valve, and wherein said fluid flow to coronary vessels of the heart is provided by a separated pump. When the pulmonary valve is closed, the third fluid flow will become zero. The fourth fluid flow through the first resistor will maintain the diastolic pressure.

In a further embodiment, the method may further comprise: measuring a pressure at the afterload device before the first resistor; interrupting said decrease of the conductance during said second part of the systolic phase when said predetermined diastolic pressure has been reached.

In a yet further embodiment, the method may further comprise: measuring a flow or a pressure at the afterload device before the first resistor; interrupting the increase of the conductance when a predetermined conductance has been reached.

In a still further embodiment, the first resistor may be controlled by a motor providing a current, which is proportional to the pressure before the first resistor and which provides said measuring of the pressure before the first resistor.

In a still, yet further embodiment, the method may further comprise: measuring a flow or a pressure at the afterload device before the first resistor and determining a start of the systolic phase when said flow or pressure exceeds a predetermined flow or pressure or increases suddenly.

In another aspect, there is provided an afterload device for providing an afterload to an outflow fluid flow expelled from a ventricle of a beating heart, comprising: a first connector for connecting the afterload device to a ventricle of the heart for receiving the outflow fluid flow expelled by the ventricle; an afterload body for receiving the outflow fluid flow from the ventricle; a valve body for forming a first resistor having an adjustable first resistance and for influencing upon the outflow fluid flow through the afterload body; a control device for receiving input data and arranged to adjust the first resistor to have at the start of a systolic phase a first conductance, which increases during a first part of the systolic phase followed by a decrease of conductance during a second part of the systolic phase; a second connector for providing a second fluid flow to the first connection between the afterload device and the ventricle whereby the second fluid flow, during a diastolic phase is divided in a third fluid flow towards the ventricle and a fourth fluid flow through the resistor; a pump for providing said second fluid flow; pressure sensors and/or flow sensors for measuring pressures and/or flow rates within at least one of: atrium, ventricle, input of the afterload device, output of the afterload device, first or second fluid flows; position meters for measuring valve body position.

In an embodiment, the afterload body may be non-elastic. This will minimize the compliance of the afterload body. The afterload body may have a small volume for providing a small inertance, whereby the volume is smaller than an expelled volume of fluid by the ventricle in a cardiac beating cycle. The volume of the afterload body may be smaller than 50 ml, such as smaller than 30 ml, for example smaller than 20 ml.

In another aspect, the afterload device is connected to the heart for receiving outflow fluid flow expelled by the ventricle during a systolic phase of a heart beat. The afterload device may comprise an afterload body, a valve body and a positioning device for positioning the valve body in the afterload body for forming an adjustable resistor. A control device receives input data, such as pressure and/or fluid flow rates and/or valve body position, and is arranged to adjust the adjustable resistor to have a first conductance at the start of the systolic phase, which increases during a first part of the systolic phase followed by a decrease of conductance during a second part of the systolic phase. An auxiliary fluid flow is provided to the connection between the afterload device and the ventricle, which is divided in a reverse fluid flow towards the ventricle and a fluid flow through the resistor. The reverse fluid flow closes the artery and/or pulmonary valves. At the left side of the heart, the reverse fluid flow provides required coronary flow.

### BRIEF DESCRIPTION OF THE DRAWINGS

Further objects, features and advantages of the invention will become apparent from the following detailed description of embodiments of the invention with reference to the drawings, in which:

FIG. 1 is a schematic view of a Windkessel diagram.

FIG. 2 is a diagram showing pressures in a working heart.

FIG. 3 is a schematic diagram of a first embodiment of the invention.

FIG. 4 is a schematic diagram of a second embodiment of the invention.

FIG. 5 is a schematic diagram of a combination of the first and the second embodiment.

FIG. 6 is a schematic diagram of a third embodiment of the invention.

FIG. 7 is a schematic diagram of a fourth embodiment of the invention.

FIGS. 8 and 9 are side views in cross-section of an embodiment of the afterload device.

FIGS. 10 and 11 are side views in cross-section and FIG. 12 is a plan view of another embodiment of the afterload device.

FIGS. 13-16 are cross-sectional views of alternative valve devices in an afterload device.

FIGS. 17 and 18 are diagrams showing arterial pressure, electric current and position of the afterload device.

### DETAILED DESCRIPTION OF EMBODIMENTS

Below, several embodiments of the invention will be described. These embodiments are described in illustrating purpose in order to disclose the best mode and to enable a skilled person to carry out the invention. However, such embodiments do not limit the scope of the invention. Moreover, certain combinations of features are shown and discussed. However, other combinations of the different features described are possible within the scope of the invention.

The present invention is an improvement of the system disclosed in U.S. Pat. No. 10,405,756B2. A drawback with the previously known system is that it is cumbersome to adjust the compliance and inertance afterload on the heart.

Embodiments of the present invention comprise a device which can adjust compliance, inertance and resistance within wide limits and which can adjust diastolic and systolic pressure limits. Before a transplantation, the heart can be exposed to many different conditions ex vivo emulating possible working conditions. The prospect of the heart to operate properly after a transplantation can be examined. The device may also be used at a heart inside the body in vivo or ex vivo, wherein the heart is isolated and connected to the device.

In addition, the present embodiments enable a specialist to study the heart ex vivo outside the body under many conditions in order to gain further knowledge of how the heart reacts to certain afterloads, flows and/or functional abnormalities, which can result in important insight that can be used for advancing the art of treatment of heart diseases and heart transplantation science.

Moreover, the heart may be exposed to drugs and/or pharmaceutical substances and/or other external stimuli and the influence on the heart may be examined.

Embodiments of the invention enable the imposition of hard limits on systolic and diastolic pressures independently of inotropic variations of the heart and for example arrhythmia. The hard limits may be decoupled from other parameters and conditions. The impedance of the afterload is dynamically adjustable and programmed to emulate a desired reference dynamic, for example in the form of a programmable resistance, compliance and inertance of the afterload.

Some prior art systems start from the so-called Windkessel model, which describes the load of the heart in a lumped model, wherein an afterload of a heart comprises the components compliance, inertance and resistance.

The present embodiments are based on the finding that compliance, inertance and resistance can be provided using a controlled resistor, which is adjusted to emulate a combination of compliance, inertance and resistance. The controlled resistor is controlled in real time by a computer to be adjusted within the normal cardiac cycle to simulate the operations of the combination of compliance, inertance and resistance or other reference dynamics as preferred by the designer or user and in addition have the ability to set hard limits on the systolic and diastolic pressures as well.

This means that the controlled resistor may have different values during the start, middle and end of a systolic heart cycle and there between. The resistor is controlled at least 10 times faster than the cardiac cycle, preferably at least 20, 40 or 100 times faster or more. If the cardiac cycle is 1 Hz (beat/second), the resistor is controlled at least by 10 Hz, 20 Hz, 40 Hz or 100 Hz or more. The expression “faster” means that the controlled resistor can be adjusted to different resistances more than one time per cardiac cycle, for example more than 10, 20, 40 times per cardiac cycle or 100 times per cardiac cycle, or more. The control may be analog or digital. If the control is digital, the system is sampled by a frequency which is more than 10 Hz, 20 Hz, 40 Hz or 100 Hz or faster.

The controlled resistor is arranged as an afterload to the heart, similar to the arrangement of the afterload according to U.S. Pat. No. 10,405,756B2.

Initially, there is discussed the operation of the afterload on the left side of the heart. Similar considerations are valid for the right side of the heart or a combination of both sides, as described below.

The afterload should be arranged as close to an aortic residue as possible to minimize any parasitic compliance, inertance and resistance before the controlled resistor that add to the total afterload. In addition, the geometry of the afterload device should add as little as possible to the inertance and compliance. The afterload device comprises an afterload body, which is non-elastic and contribute as little as possible to the total compliance. Moreover, the afterload body has as small volume as possible in order to add as little as possible to the inertance. It is desired that the volume of the afterload body is smaller than an ejection volume of the heart, which often is given as 50 or 70 ml. A volume of less than 70 m¹⁵⁰ ml or 30 ml would be appropriate, such as 20 ml or smaller.

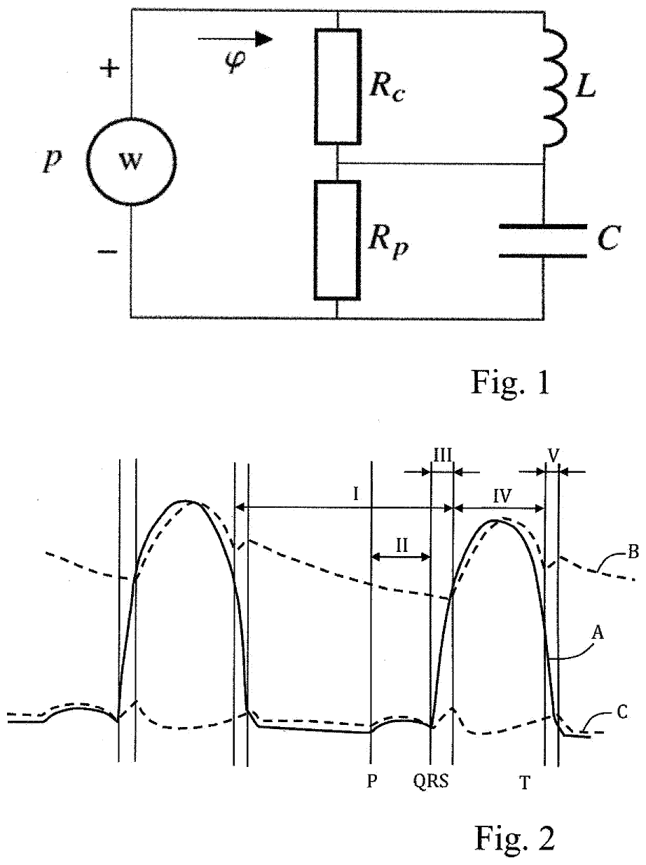

In an equivalent electric scheme, the components compliance, inertance and resistance can be replaced by capacitance, inductance and resistance as shown in FIG. 1.

Any system built up as a network of resistors, capacitors and inductors can be presented in linear state space form as

$$\dot{x} = Ax + Bu , \qquad ( 1 )$$

$$y = Cx + Du ,$$

where x is the internal system state, u is some controllable input and y is some measurable output. Within the electrical analogy u and y are scalars or vectors, where each element is either a voltage or a current or linear combinations thereof.

We note that there is an equivalence analogy between hydraulic and electric systems, where electric resistance, capacitance, and inductance correspond to volumetric flow resistance, compliance, and inertance. Electric potential (voltage) corresponds to hydraulic pressure, and electric current corresponds to volumetric flow.

Inertance is a measure of the pressure difference in a fluid required to cause a unit change in the rate of change of volumetric flow-rate with time.

Since Windkessel models are networks of the aforementioned component types, they can be represented on the form (1). Within the classical formulation, the input u=φ is aortic volumetric flow, and the output y=p is aortic pressure.

A concrete example is provided by the 4-element parallel Windkessel model, that can be represented as the circuit diagram of FIG. 1, and described in state space form as

$$\dot{x} = [ \begin{matrix}\frac{- 1}{{CR}_{p}} & 0 \\ 0 & \frac{{R}_{c}}{L}\end{matrix} ] x + [ \begin{matrix}1 \\ {R}_{c}\end{matrix} ] φ , \qquad ( 2 )$$

$$p = [ \begin{matrix}\frac{1}{C} & \frac{- {R}_{c}}{L}\end{matrix} ] x + [ {R}_{c} ] φ .$$

The Windkessel model parameters are peripheral resistance (Rₚ) representing the resistance of the vasculature, arterial compliance (C), characteristic resistance of the great vessel (R_{c}) (the aorta for the left side and pulmonary artery for the right side), and the inertance of the flowing volume (L).

For our modeling purposes it is more natural to consider instantaneous cardiac power (current times voltage drop), or equivalently volumetric flow times pressure drop, as input. This is a more natural approach, since flow and pressure are dynamically coupled to afterload dynamics, while the instantaneous power is closely related to the inotropy and is consequently easier to observe in isolation from other signals. Instead of the linear time-invariant (LTI) ordinary differential equation (ODE) of (1), we then need to deal with the equivalent nonlinear differential algebraic equation (DAE):

$$\dot{x} = Ax + B φ , \qquad ( 3 )$$

$$w = φ p ,$$

$$p = Cx + D φ .$$

We can think of (3) as a Windkessel model with power as input. However, it describes exactly the same dynamics as its ODE counterpart with volumetric flow as input.

Now let us replace the Windkessel dynamics with a time-varying resistor R to obtain:

$$φ = \frac{p}{R} . \qquad ( 4 )$$

Confining to the case w>0 (the heart exerting power, i.e. the systolic part of the cardiac cycle), we can combine (4) with w=φp to obtain:

$$p = \sqrt{R w} \qquad ( 5 )$$

The conclusion from (5) is that we can shape any p>0 from any w>0 (where the inequalities are point-wise in time), and thus recreate any LTI dynamic relation (including capacitive ones) between w and p, or, equivalently, between φ and p.

Given a w or φ signal of choice, we can simulate (3) or (2) to obtain the corresponding p when the heart is exerting power. Then, the desired R at any given time is obtained through (5) or (4).

FIG. 1 is a circuit diagram of an equivalent of the 4-element parallel Windkessel model. The heart is modeled as a power source w, as opposed to the flow (current) source utilized in the classic Windkessel formulation.

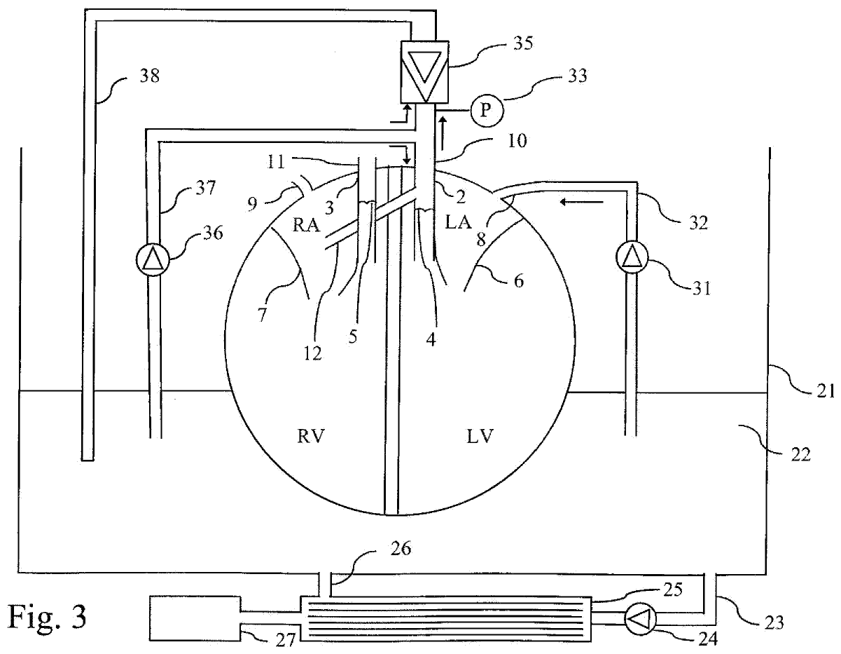

However, there are further problems to take into consideration. In order to fully understand the heart function, a normal cardiac cycle of a human being (or any animal with a comparable heart physiology) is described with reference to FIG. 2. The diagram shows the left intraventricular pressure A, the aortic pressure B and the left atrial pressure C. The cycle looks similar for the right side of the heart, however, the pressures are about six times lower.

During diastole, period I, blood flows continually into the heart from the great veins and into the atria. About 80% of the blood flows directly through the atria into the ventricles.

The filling pressure of the atria is normally 0 to 15 mmHg (0 to 20 cm H₂O). The filling pressure determines the amount of blood flowing into the ventricles and the heart pumping (Frank-Starling mechanism).

At onset of a P-wave, the atria contract and fill the ventricles with an additional 20% of blood (period II).

About 0.16 second after the onset of the P-wave, QRS waves appear and initiate contraction of the ventricles and causes the ventricular pressures to begin rising. Immediately after ventricular contraction begins, the ventricular pressure rises abruptly causing the atrial-ventricular A-V valves to close. About 0.02 to 0.03 seconds are required for the ventricles to build up sufficient pressure to push the semilunar (aortic and pulmonary) valves open against the pressure in the aorta and pulmonary artery. This is the period of volumic or isometric contraction (period III).

Then, blood is ejected out of the ventricles (systole, period IV), about 70% of the blood emptying occurring during the first third of the period of ejection (period of rapid ejection) and the remaining 30% emptying during the next two thirds (period of slow ejection).

At the end of the systole, ventricular relaxation begins suddenly, allowing both the right and left intraventricular pressure to decrease rapidly (ventricular T-wave). The elevated pressure in the distended large arteries that have just been filled with blood from the contracted ventricles immediately push blood back toward the ventricles, which snaps the aortic and pulmonary valves closed.

For another 0.03 to 0.06 second, the ventricular muscle continues to relax, even though the ventricular volume does not change, giving rise to a period of isovolumic or isometric relaxation (period V). During this period, the intraventricular pressures decrease rapidly back to their low diastolic levels. Then, the A-V-valves open to begin a new cycle.

During diastole, normal filling of the ventricles increases the volume of each ventricle to about 110 to 120 ml. This volume is called the end-diastolic volume. Then, as the ventricles empty during systole, the volume decreases by about 70 ml, which is called the stroke volume output. The remaining volume in each ventricle, about 40 to 50 ml is called the end-systolic volume. The fraction of the end-diastolic volume that is ejected is called the ejection fraction, usually equal to about 60%.

The systole lasts about 30 to 45% of the total cardiac cycle.

Blood also passes through the coronary vasculature to provide nutrition and oxygen to the heart muscle. During the high pressure systolic phase, the coronary blood flow is lower than during the low pressure diastolic phase. The reason for this is strong compression of the ventricular muscle around the intramuscular coronary vessels during systolic contraction. During diastole, the cardiac muscle relaxes and no longer obstructs blood flow through the coronary vasculature.

Both coronary arteries start from a position at the aortic root just beyond the aortic valve. The left coronary artery supplies mainly the anterior and left lateral positions of the left ventricle, whereas the right coronary artery supplies most of the right ventricle as well as the posterior part of the left ventricle. Both the right and left coronary arteries start from the aortic root and open to the right atrium. A very small amount of coronary venous blood also flows back into the heart through very minute thebesian veins, which empty directly into all chambers of the heart.

The resting coronary blood flow in a human being is about 3.75 ml/sec (225 ml/min) which is about 4 to 5% of the total cardiac output, which is about 83 ml/sec (5 l/min).

At the end of a systole, the blood flow is reversed to push blood back toward the ventricles, which snaps the aortic and pulmonary valves closed. If the components of the Windkessel model according to FIG. 1 are replaced by a controlled resistor, there is no or minimal compliance (capacitance) that can push blood back (reverse the flow) and close the aortic or pulmonary valve.

Another complication is that the aortic root and the connection of the aorta to a controlled resistor necessarily comprise a volume of blood having an inertance (inductance) and compliance (capacitance). This parasitic compliance and inertance should be minimized. Moreover, the compliance and inertance of the afterload device per se should be minimized.

According to the present embodiments, the ability to generate reverse flow, and thus compliance is created by adding an auxiliary flow source to the circuit between the aortic (or pulmonary) valve and the controlled resistor. The auxiliary flow may operate during the whole cardiac cycle, both during diastole and during systole. Alternatively, the auxiliary flow operates during the diastolic phase and optionally during at least a part of the systolic phase.

The auxiliary flow is equal or larger than the expected coronary flow, for example twice the coronary flow (left portion of the heart). The auxiliary flow has a sufficient overhead to emulate the compliant and other not purely resistive properties of the desired vascular dynamics.

For the right portion of the heart, coronary flow is provided separately. In this case the auxiliary flow may be provided only during a period when the pulmonary valve should close and for maintaining a sufficient diastolic pressure during the diastolic phase. As for the left portion of the heart, the auxiliary flow may be provided both during diastole and optionally during systole.

During the end of the systole, the aortic pressure decreases and when the aortic pressure reaches a predetermined pressure corresponding to the desired start-diastolic pressure, the controlled resistor is controlled to prevent further decrease in pressure, for example by decreasing its conductance. This causes the flow through the resistor to decrease and the auxiliary flow, which during systole was passing through the resistor, now divides into a small flow (or no flow) through the resistor and a “reverse” flow towards the ventricle. The reverse flow results in the closure of the aortic valve (and the pulmonary valve). The controlled resistor is continuously adjusted to divide the auxiliary flow between the flow shunted through the controlled resistor and the reverse flow that maintains the desired diastolic pressure despite volume lost to coronary flow. This is done by successively adjusting the resistance of the controlled resistor during diastole to generate a pressure as indicated by curve B in FIG. 2 during period I, diastole, or another curve according to the programmed dynamics.

The auxiliary input flow may have a flow rate which is controlled by a separate auxiliary pump. The auxiliary input can be controlled to provide for example a constant flow rate, for example 450 ml/min (double the expected coronary flow), or a constant pressure or any combination thereof. It is required that the auxiliary flow is at least equal to the peak coronary flow and that the pressure of the auxiliary flow is at least equal to the start-diastolic pressure.

When modeling the auxiliary input as a pressure source, it can be understood within the aforementioned electrical analogy as adding an offset to the potential at the interface between the aorta and the controlled resistor. This enables negative pressures and thus negative flows, with respect to offset level, which gives the device the capability to emulate compliant behavior, as well as a range of active dynamics that would not be attainable with only passive components such as the resistor, inductor, and capacitor that make up the electrical analogy of the Windkessel model.

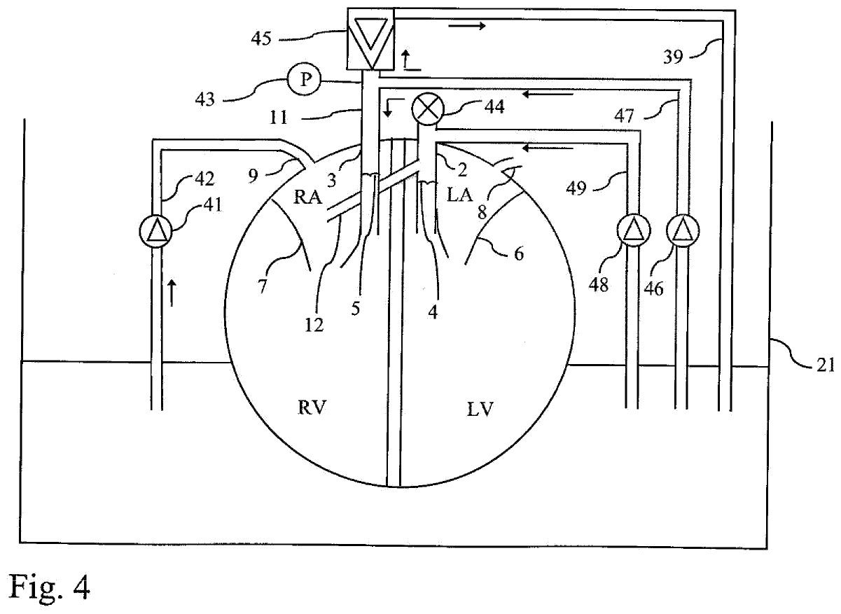

A first embodiment of the invention is disclosed in FIG. 3, which is a schematic diagram. The heart is shown schematically to comprise a left atrium, LA, a left ventricle LV, a right atrium RA, and a right ventricle, RV. The heart is normally shown inverted with the left items to the right and the right items to the left.

The heart 1 is harvested with an aortic residue 2 and a pulmonary artery residue 3. The aortic residue 2 ends in an aortic valve 4 and the pulmonary artery residue 3 ends in a pulmonary valve 5. The ventricles LV and RV are connected to the atria LA and RA via atrial-ventricular A-V-valves 6 (mitral valve) and 7 (tricuspid valve). The atria are connected to inlet tubes 8 and 9, respectively, and the aortic and pulmonary arteries 2 and 3 are connected to outlet tubes 10 and 11, respectively.

A coronary vessel 12 (including both coronary arteries) starts at a position slightly beyond the aortic valve 4 and ends in the right atrium RA.

The heart 1 is arranged in a vessel 21 comprising a fluid 22 to be circulated through the heart. The heart may be hanging above the surface of the fluid, or resting on a supportive platform or partially immersed in the fluid as shown in FIG. 3.

The vessel 21 comprises an oxygenation outlet 23 connected to an oxygenation pump 24, which pumps the fluid 22 in a circuit through an oxygenator 25 and back to the vessel via oxygenation inlet 26. The fluid may be temperature controlled via a heater/cooler unit 27 connected to the oxygenator. The oxygenator is connected to a source of gas, such as oxygen and carbon dioxide as is conventional. In this manner, the fluid 22 in vessel 21 is continuously oxygenated. The oxygenation device may be used in any one of the disclosed embodiments of the afterload device.

The fluid may be whole blood or Krebbs solution.

A circulation pump 31 pumps fluid via a circulation tube 32 from the vessel 21 into the inlet tube 8 and to the left atrium LA and further to the left ventricle LV, thereby filling the left atrium LA and the left ventricle LV during a first portion of the diastole, whereby the aortic valve 4 is closed and the mitral valve 6 is open.

An afterload 35 is connected to the outlet tube 10 from the aortic residue and conducts the fluid flow from the aortic root, through a controlled resistor of the afterload and further back to the vessel 21 via an outlet tube 38. The afterload 35 will be described in further details below.

A pressure meter 33 is arranged at the inlet of the afterload. Additional pressure meters may be arranged at other positions, for example after the afterload and at the inlet tube 8 and inside the atria and/or ventricles.

An auxiliary pump 36 is connected to the aortic residue and pumps fluid via an auxiliary tube 37 from the vessel 21 to the connection between the aortic root and the afterload.

The operation of the embodiment is the following: Circulation pump 31 continuously pumps fluid at a normal rate of for example 83 ml/sec corresponding to about 5 l/min.

The auxiliary pump 36 pumps auxiliary fluid at a rate of for example 450 ml/min, which is approximately double the expected coronary flow. The auxiliary flow rate is not critical but need to be sufficiently large.

The auxiliary flow rate is provided to emulate the compliant and other not purely resistive properties of the desired vascular dynamics.

In addition, for the left part of the heart, the auxiliary flow provides coronary flow during diastole.

At the start of a systole (between period III and period IV in FIG. 2), the heart pumps fluid in an outflow fluid flow from the ventricle through the aortic valve and out to the controlled resistor in the afterload 35. Since the systolic phase is approximately one third of the total cycle, the mean flow out of the ventricle may be more than about 15 l/min, which passes through the controlled resistor together with 450 ml/min from the auxiliary flow (a small portion passes through the coronary vessel 12).

The controlled resistor is adjusted so that a desired afterload dynamics is obtained. This can be according to the Windkessel diagram of FIG. 1 or any other reference dynamics, see further below. During the period IV (systole), the heart's ability to produce pressure and flow can be studied.

After the systolic pressure has reached its maximum pressure (for example 120 mmHg), which is dependent on the heart, the filling of the ventricle, and the afterload, among others, the pressure starts to decrease and when the pressure in the ventricle drops below the aortic pressure the closure of the aortic valve results. Thereafter, the resistance of the controlled resistor is adjusted so that the desired aortic diastolic pressure is obtained, for example a slow diastolic pressure decrease as shown in curve B of FIG. 2.

The auxiliary flow may be continuous, variable or intermittent, which may be obtained by variable pump speed or valves and/or resistors. In the continuous flow case, the instantaneous resistance of the afterload in combination with the instantaneous pressure in the ventricle determines the amount of the flow that is shunted away from the heart, such that more flow is shunted away from the heart during systole (low coronary flow and flow is augmented by the flow from the ventricle) while more flow is directed towards the heart during diastole to account for coronary flow and compliance in the desired dynamics. As an alternative to shunting parts of the flow away from the heart, the quantity of flow could be equivalently reduced during such periods (either by variable auxiliary pump flows, or by intermittent auxiliary pump flows) requiring less variation in the afterload resistance to account for these changes in the quantity of reverse flow.

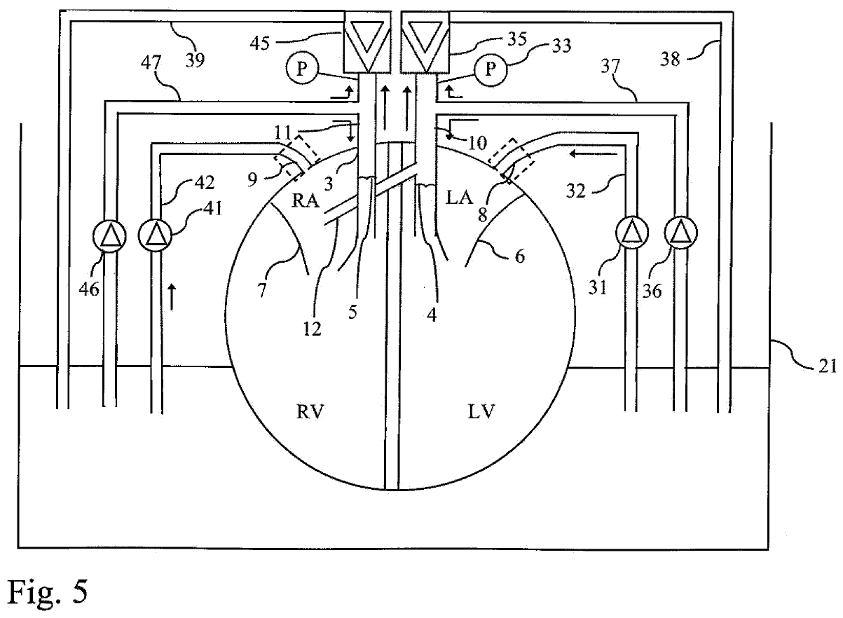

FIG. 4 is a schematic diagram similar to FIG. 3 and shows a similar arrangement on the right side of the heart.

A circulation pump 41 pumps fluid via a circulation tube 42 from the vessel 21 into the inlet tube 9 and to the right atrium RA and further to the right ventricle RV, thereby filling the right atrium RA and the right ventricle RV during a first portion of the diastole, during which the pulmonary valve 5 is closed and the triscupid valve 7 is open.

An afterload 45 is connected to the outlet tube 11 and conducts the fluid flow from the outlet tube 11, through a controlled resistor of the afterload 45 and further back to the vessel 21 via an outlet tube 39. A pressure meter 43 is arranged at the inlet of the afterload.

An auxiliary pump 46 is connected to the outlet tube 11 and pumps fluid via an auxiliary tube 47 from the vessel 21 to the connection between outlet tube 11 and the afterload 45.

There is also arranged a coronary flow to the aortic root, which is provided by a separate coronary pump 48, which pumps fluid from vessel 21 via a coronary tube 49 to the outlet tube 10 leading to the aortic root. The distal end of outlet tube 10 is closed by a valve 44. The coronary flow pump 46 is adjusted to obtain the required coronary flow.

The operation of the embodiment is the following: Circulation pump 41 continuously pumps fluid at a normal rate of for example 5 l/min (83 ml/sec). The auxiliary pump 46 pumps fluid at a small rate of for example 180 ml/min (3 ml/sec). Because no coronary flow is required, the auxiliary flow may be small, only sufficient to emulate the compliant and possibly other non-resistive properties of the desired vascular dynamics and generate a reverse flow or negative flow to close the pulmonary valve at start-diastole.

The controlled resistor is adjusted by a computer so that a desired afterload is obtained throughout the cardiac cycle. This can be according to the Windkessel diagram of FIG. 1 or any other reference dynamics.

There are hard limits imposed on systolic and diastolic pressures, which are achieved by stopping conductance increase once the systolic pressure is reached and decreasing conductance once the diastolic pressure is reached. In this context the auxiliary flow is essential, as just decreasing conductance cannot maintain coronary perfusion pressure unless there is a pressure (potential, voltage) at the connection point between aorta and the afterload device.

There is a risk that the large flow from pump 41 via tube 42 forms a direct flow through the atrium and ventricle and keep the pulmonary valve open. In order to counteract this phenomenon, a vortex device, forming a compliance or capacitance may be arranged at the inlet of connection 9 (and inlet of connection 8) as shown in U.S. Pat. No. 10,405,756B2. The same arrangement may be used in the embodiment according to FIG. 3 or any of the other embodiments.

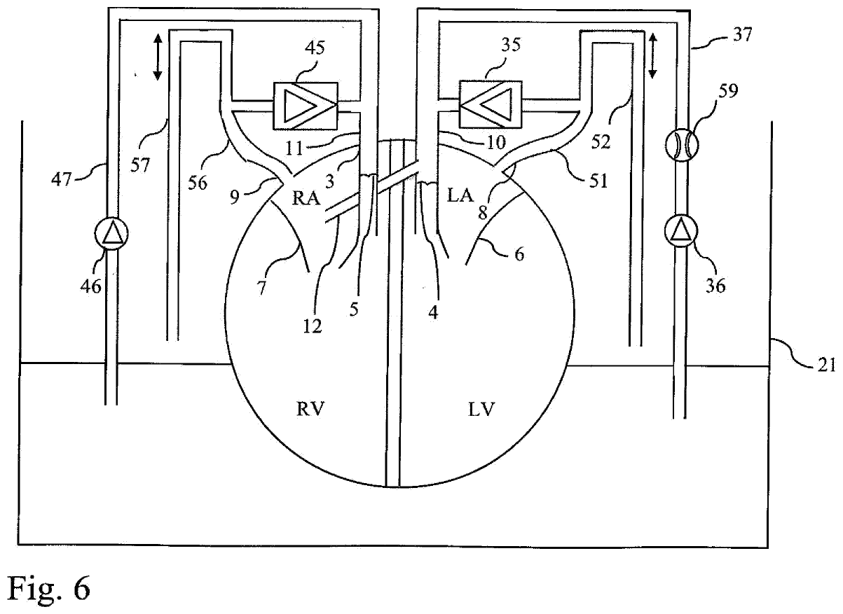

The two embodiments of FIGS. 3 and 4 may be combined in order to investigate both sides of the heart simultaneously, for example in parallel. This is shown in FIG. 5, wherein the same components as in FIGS. 3 and 4 have the same reference numerals. The operation of the embodiment of FIG. 5 is the same as described above.

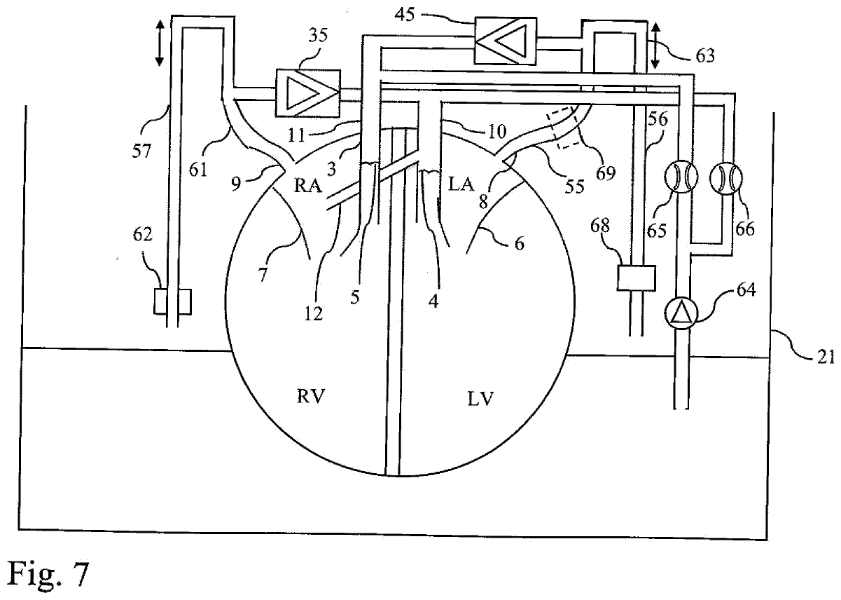

In the human body, the outflow from the ventricle is passed back to the inlet of the atrium via the vascular dynamics of the body. This may be studied in the embodiment shown in FIG. 6 (still in parallel).

In the embodiment of FIG. 6 the circulation pump 31 of the embodiment according to FIG. 3 is replaced by the outflow from the corresponding controlled resistor. However, fluid is added continuously by the auxiliary pump 36 (and 46) resulting in that a corresponding amount of fluid should be removed per heart cycle.

FIG. 6 shows a schematic diagram of an embodiment in which the fluid outflow from the afterloads of the embodiments of both FIG. 3 and FIG. 4 are connected to the inlet tubes 8 and 9, respectively. Fluid outflow from outlet tube 10 passes through the afterload 35 and further to an outflow tube 51, whereupon the fluid enters the inlet tube 8 to the left atrium LA. A small flow is diverted from the outlet tube 51 via an overflow tube 52 arranged at a position higher than the afterload. The overflow tube 52 ends before the surface of the fluid in vessel 21 as shown.

Any excess fluid added by the auxiliary pump 36 pours over the overflow tube 52 and is given off to the vessel. The height of the overflow tube 52 may be adjustable to adjust the inlet pressure, which may be in the range of a few mmHg, such as 3 to 15 mmHg, corresponding to 4 to 20 cm water pillar.

The right side of the heart may be arranged in a similar manner: Fluid outflow from outlet tube 11 passes through the afterload 45 and further via an outlet tube 56 to the inlet tube 9 to the right atrium RA. A small flow is diverted from the outlet tube 56 via an overflow tube 57 arranged at a position higher than the afterload. Overflow tube 57 ends before the surface of the fluid in the vessel 21 as shown.

Any fluid added by the auxiliary pump 46 pours over the overflow tube 57 and is given off to the vessel. In this manner a substantial constant inlet pressure is present at inlet tube 9. The height of the overflow tube 57 may be adjustable to adjust the inlet pressure, which may be in the range of a few mmHg, such as 1 to 10 mmHg, corresponding to 1.5 to 14 cm water pillar.

The auxiliary pump 36 of the embodiment of FIG. 6 may alternatively be operated as a constant pressure pump, that delivers a constant pressure of for example 5 to 10 mmHg above the predetermined diastolic pressure or even a pressure exceeding the maximum systolic pressure. Moreover, the auxiliary tube 37 may be provided with a restriction 59, which may be arranged so that a desired coronary flow is obtained during diastole. During systole, there may be a small negative flow through restriction 59 since the pressure to the left of the restriction may be larger than to the right of the restriction. When the pressure at the aortic root approaches the diastolic pressure, the flow through restriction 59 is reversed, which may be measured by a pressure monitor or flow monitor. At the same time the controlled resistor 35 is adjusted to a large resistance, which means that the flow through the restriction 59 will pass towards the aortic valve and eventually close the aortic valve. Finally, the controlled resistor is controlled to provide the desired coronary flow at the desired diastolic pressure. This embodiment of the auxiliary flow device may be used in any of the disclosed system embodiments.

Other embodiments of the auxiliary flow may be that the auxiliary flow is controlled by a valve and/or combinations of volume-controlled and pressure-controlled auxiliary flow.

The concept of auxiliary flow is to arrange that a positive flow is obtained through the controlled resistor during diastole and optionally during systole. During diastole, the auxiliary flow is divided in a flow through the controlled resistor and a flow to the coronary vessels. In this manner, the controlled resistor can adjust diastolic pressure by influencing this division.

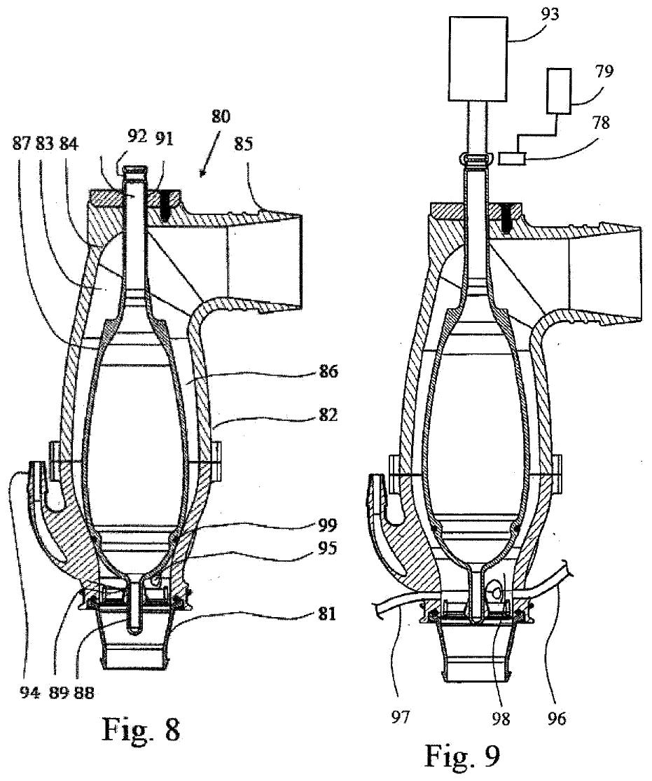

In the embodiment of FIG. 6, the two sides of the heart operate in principle independently of each other, i.e. in parallel. FIG. 7 is a schematic view of an embodiment in which the two sides of the heart are connected in the same manner as in the living body, i.e. in series.

In the embodiment of FIG. 7, fluid outflow from outlet tube 10 from the aortic root passes through the afterload 35 and further to a return tube 61 and enters the inlet tube 9 of the right atrium RA. An overflow tube 57 is connected to the return tube 61. The overflow tube 57 has at its top a curvature of 180 degrees and passes down and ends shortly above the fluid level of vessel 21. Any fluid added by the auxiliary pump pours over the overflow tube 57 and is given off to the vessel. Before the end of tube 57, there is arranged a flow meter 62, which measures the outflow rate that pours over the overflow tube 57. The height of the overflow tube 57 may be adjustable. A crosswise flow takes place from the outlet of the left ventricle to the inlet of the right atrium.

The right side of the heart is arranged in a similar manner: Fluid outflow from outlet tube 11 passes through the afterload 45 and further to a return tube 55 and enters the inlet tube 8 to the left atrium LA. An overflow tube 63 has at its top a curvature of 180 degrees and ends shortly above the fluid level of vessel 21. Any excess fluid added by the auxiliary pump pours over the overflow tube 63 and is given off to the vessel. The height of the overflow tube 63 may be adjustable. Moreover, an oxygenator 69 may be inserted between the outlet of the controlled resistor 45 and the inlet tube 8, simulating the lungs in a human, but the device may be operated without this oxygenator. If the oxygenator 69 is included, the oxygenation arrangement 23 to 27 in FIG. 3 is no longer required.

Before the end of tube 56, there may be arranged a flow meter 68, which measures the outflow rate that pours over the outflow tube 63. The height of outflow tube 63 is adjustable to adjust the inlet pressure. A crosswise flow takes place from the outlet of the left ventricle to the inlet of the right atrium.

The auxiliary flow is generated by a constant pressure pump 64 that generates a pressure higher than the systolic pressure in the left portion of the heart. The pump 64 is connected to the aortic root via a first restriction 65. In addition, pump 64 is connected to the outlet tube 11 from the right ventricle via a second restriction 66. The second restriction can be larger than the first restriction. It should be noted that the pressures at the right side of the heart are approximately only one sixth of the pressures at the left side. The restrictions may be fixed and have constant resistance.

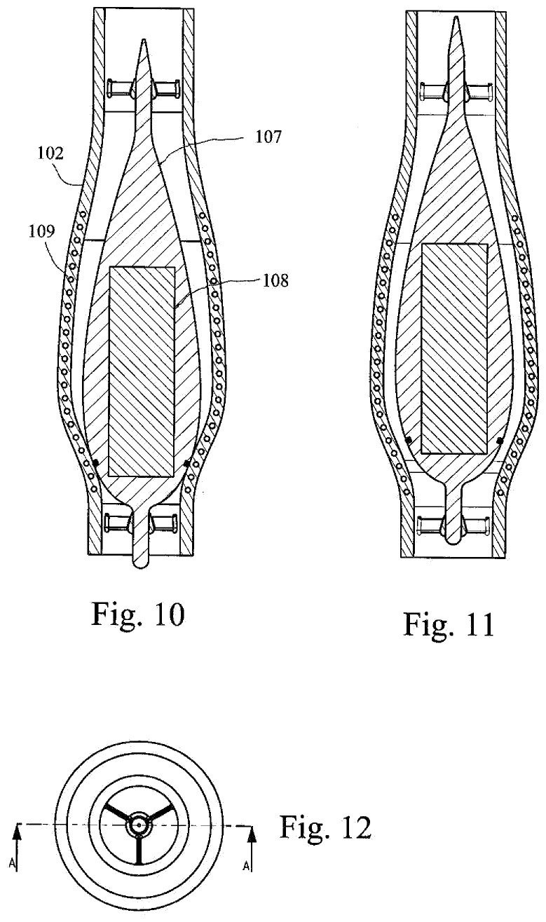

FIGS. 8 and 9 show a controlled restrictor forming a resistance to be used as an afterload. The afterload 80 comprises an inlet piece 81 connected to an afterload body 82 with a substantially cylindrical cross-section, Inside the cylinder there is formed a slightly curved inner tubular space 83 which matures in a curvature 84 over 90 degrees and is connected to an outlet piece 85. Inside tubular space 83, there is arranged a valve body 87 having a shape approximately corresponding to the tubular space 83 but leaving a distance 86 to the tubular space 83. The valve body 87 is moveable along the tubular space between a first closed position shown in FIG. 8 to an open position shown in FIG. 9. The valve body 87 is moved by a linear motor 93 schematically shown in FIG. 9.

The auxiliary flow is added via an inlet tube 94 that opens at an opening 95 to the tubular space 83 in an area 98 slightly below the valve body 87 when in its lowest position. The afterload device further comprises pressure tubes 96, 97 that connect to the area 98 below the valve body 87 for connection of pressure meters to the afterload device.

In the above specification, specific direction references are indicated, such as above, below etc. Such references relate only to the embodiments as shown in the drawings.

The valve body 87 comprises a guiding pin 88 at the bottom end that is inserted in a central hole 89 in the afterload body 82. Another guide pin 90 is arranged at the opposite end of the valve body. The guide pin 90 passes out through the afterload body through a hole 91 and ends in a flange 92 that is connected to the linear motor 93. The guide pin 90 is sealed by a sealing.

The valve body 87 is hollow to reduce mass and inertance.

An O-ring 99 is arranged at the bottom of the valve body 87 for sealing against the afterload body.

The linear motor 93 comprises electric coils that are connected to a control device for moving the valve body up and down, whereby the resistance of the controlled resistor is adjusted. In addition, there is arranged a position meter 78 that measures the exact position of the valve body 87 in relation to the afterload body 82. The position of the valve body controls the conductance of the afterload device for flow there through.

Data measuring for example the position and/or the pressures and/or the flow rates are fed to a computer 79, that calculates a control signal to the linear motor 78, for adjusting the controlled resistor position.

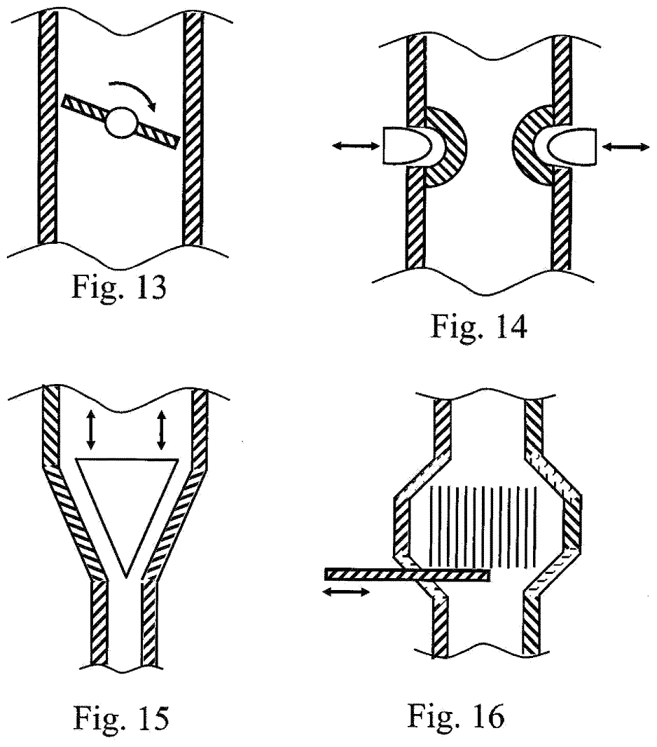

FIGS. 10, 11 and 12 shows another embodiment of the afterload device, in which the linear motor is built into the afterload and valve body. Valve body 107 comprises a permanent magnet 108 centrally arranged in the valve body. In the afterload body 102 there is arranged a coil 109 of electric wires encircling the valve body. When current is passed through coil 109, a magnetic force is generated, which pushes the magnet and the valve body up or down. Thus, the position and force of the valve body 107 may be precisely controlled.

The driving coils in this, as well as other embodiments of the device can also act as sensors. Measuring the resistance of the coil can be used for thermal management. Measuring the current through the coil can be used as a proxy for the blood pressure applied to the plunger. By splitting the coil into an upper and lower half and superimposing a high-frequency voltage signal onto the driving voltage, it is possible to estimate the position of the magnetic core of the plunger, and thus the plunger itself, through the relative coupling between each coil half and said magnet. In this context high frequency is any signal that changes faster than the mechanical bandwidth of the plunger, being defined by its mass.

In all embodiments, flow meters may be added to measure flow rates at different portions of the device. It may be relevant to measure the outflow from left and right ventricle, auxiliary flow (or flows), flow through the controlled resistor(-a), overflow in tubes 52, 57 and 63.

Flow may be measured using an ultrasonic transducer tipped catheter by analyzing the Doppler shift in the reflected ultrasound waves. Ultrasound based time-of-flight sensors may also be used, whereby the time taken for an ultrasound pulse to travel between two transducers is used to deduce fluid velocity. Continuous thermodilution may also be used to measure blood flow, whereby a catheter with two thermistors and a small heating element cooperate to monitor changes in fluid temperature between the two transducers. Furthermore, the pressure drop over a known resistance may be used to calculate flow.

It may be relevant to measure pressure just before the controlled resistor, in the left and right atria, and in the left and right ventricles. It may be relevant to measure the position of the valve body in the afterload and the current required to keep the valve body in the desired position. Data from these measurements, i.e. pressures, flows and valve positions, may be input to a computer, that calculates the control strategy for the controlled resistor. The computer outputs position data to the linear motor 93 that adjusts the position of the valve body. The position is measured as well as the current or power needed to maintain the valve body in the calculated position. The position is related to the conductance of the controlled resistor according to a predetermined relationship. The position is approximately proportional to the conductance of the controlled resistor.

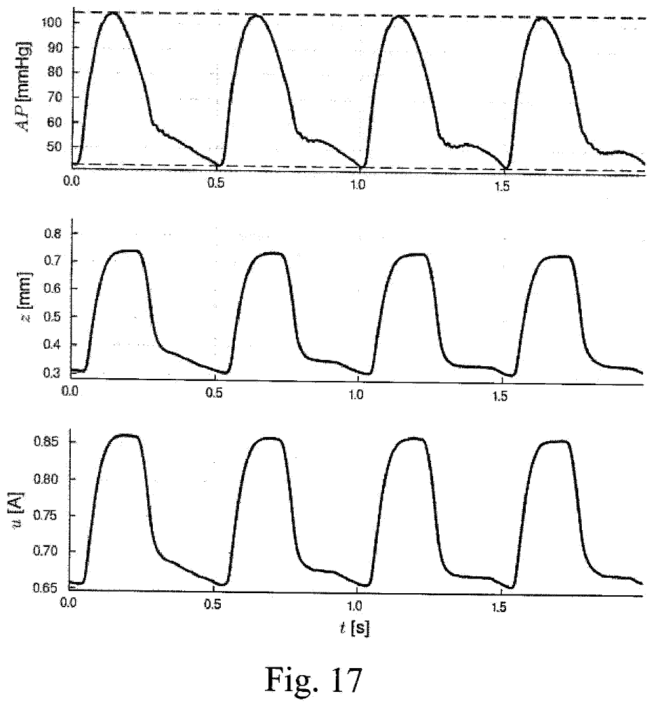

Further embodiments of the afterload device are shown in FIGS. 13-16.

FIG. 13 shows a controlled resistor, in which a disk is arranged pivotable around a horizontal shaft. The angular position of the disk is adjustable as indicated by an arrow.

FIG. 14 shows a controlled resistor, in which the walls of a tube may be influenced upon to cause a constriction of the flow area to provide a controlled resistor.

FIG. 15 shows a controlled resistor, in which a triangular or conical body cooperates with a correspondingly arranged wall of a tube. The body is moveable in the vertical direction to adjust the flow area of the arrangement.

FIG. 16 shows a controlled resistor having numerous flow channels. A disk is arranged to close some or all of the flow channels to control the total flow area.

In all embodiments, it is required to expel air before the start of a procedure. This is accomplished by starting the auxiliary flow pumps at a slow rate and pumping fluid successively through the respective tubes. Openings at the highest portion of the device may be provided with ports, through which air may be expelled or sucked out.

The fluid to be used may be whole blood; blood diluted with Krebb's solution to a desired lower hematocrit; gassed asanguinous fluids; or a solution comprising an artificial oxygen carrier such as perfluorochemical agents. The fluid should comprise nutrients sufficient to support muscle activity at least during the experiment time. Hormones may be added, such as adrenaline or nor-adrenaline etc.

The procedure is started by Langendorff perfusion, with the auxiliary flow and afterload working together as a flow-controlled pressure source and no flow supplied to the atrium (empty beating). This is done to flush the heart of possible cardioplegic solution and establish a stable rhythm before loading the heart (commencing working mode).

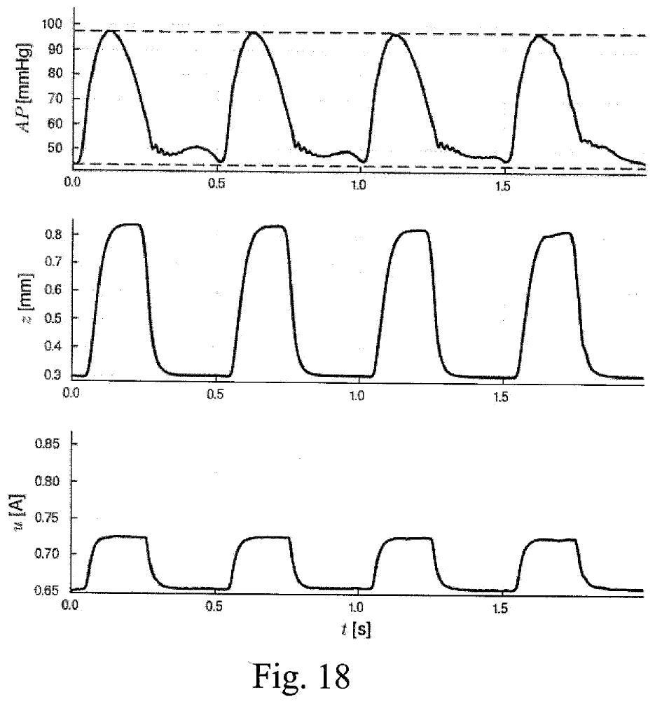

FIG. 17 shows measured experimental data using the embodiment shown in FIG. 3 with a porcine heart. Three measurements are plotted against time in seconds: aortic pressure AP; valve body position z where z=0 is the fully closed position and positive displacement indicates a more open valve position; and linear motor current u which is proportional to the closing or balancing force exerted by the motor on the valve body. As a simple example, the afterload dynamics in FIG. 17 are controlled with only valve body position feedback u=ƒ(z) such that the motor behaves as an ideal spring where u is proportional to z according to a positive real constant k. Additionally, a lower current bound uₘᵢₙ is set such that the equation governing the control of the afterload is

$$u = kz + {u}_{min} . \qquad ( 6 )$$

Time t=0 shows the transition from end-diastole to start-systole in the cardiac cycle. Before the ventricle contracts, the end diastolic pressure is approximately 45 mmHg. The aortic valve is closed, and the auxiliary flow is being divided between a flow through the coronary vessel 12 and a flow through the afterload body 86 to the vessel 21. The valve body is positioned slightly above the fully closed position; this position is defined by the force balance caused by motor current u and the flow pushing against the valve body. This results in the observed end-diastolic pressure according to equation (4), with R=ƒ(z) where the function ƒ defining the relationship between valve body position and the volumetric flow resistance of the afterload.

As the ventricle contracts, just after t=0, the pressure in the ventricle exceeds the pressure in the aorta and fluid is ejected in an outflow fluid flow from the heart into the aorta after opening of the aortic valve. During contraction, flow through the coronary vessels is negligible; both auxiliary flow and flow from the ventricle are directed through the afterload resistor. The flow from the ventricle through the afterload causes the pressure to rise. There may be a slight delay between the increase in pressure and the increase in valve body position caused by friction in the afterload mechanism and parasitic inertance. The increased flow through the afterload causes a greater upward force on the valve body, causing displacement of the valve body to a more open position and increasing conductance or lowering afterload resistance R. This displacement leads to increased current through the motor according to equation (6), resisting further displacement of the valve body.

After aortic pressure peaks, flow from the ventricle begins to drop leading to a drop in aortic pressure. As pressure in the ventricle falls below the aortic pressure and the aortic valve closes, there is a downward displacement of volume. This coincides with a negative displacement of the valve body, towards the closed position at approximately t=0.25. As in the start-systole case, there is a slight delay between the decrease in aortic pressure and the decrease in valve body position caused by friction in the afterload mechanism and parasitic inertance. The valve closure is visible in both AP and z as a sharp change in slope, referred to as the dicrotic notch (as seen in FIG. 2, V).

During diastole, fluid flows from the auxiliary flow sources are divided into a flow through the coronary vessels and a flow through the afterload. The resistance of the afterload is lower than the resistance of the coronary arteries, so the resulting pressure in the aorta is dominated by the fluid flow through the afterload resistance. In this simple case where the afterload is programmed to behave like a spring according to equation (6), compliant behavior is absent from its dynamics. However, pressure decay is still observed during diastole.

In this simple control case, the end diastolic pressure can be adjusted by changing k or uₘᵢₙ, or by changing the auxiliary flow rate; increasing any of the three will result in an increase in end-diastolic pressure. However, depending on the ability of the heart, such a change could also result in a higher peak systolic pressure as the u for all z will increase.

Non-linear behavior such as upper and lower pressure bounds can be enforced. FIG. 18 shows measured data from the same experiment as FIG. 17 with an upper limit on current, u_{systolic limit}. Motor current control (6) becomes

$$u = min [ ( kz + {u}_{min} ) , {u}_{systolic limit} \} . \qquad ( 7 )$$

As a result, while diastolic pressure remains at approximately 45 mmHg, u_{systolic limit} is reached during systole, allowing further displacement of the valve body towards the open position, leading in turn to a lower afterload resistance and a lower peak systolic pressure: about 95 mmHg compared to about 105 mmHg without the current limit as seen in FIG. 17.

A lower limit, u_{stastolic limit}, can similarly be applied to affect diastolic pressure. Due to the addition of flow from the ventricle through the afterload during systole, u_{systolic limit} may be less than u_{diastolic limit} in order to achieve the desired upper and lower pressure limits. Therefore, each limit must be applied during their respective phase of the cardiac cycle. Continuing with the simple spring control dynamics, (7) becomes

$$u = \{ \begin{matrix}min \{ ( kz + {u}_{min} ) , {u}_{systolic limit} \} , during systole , \\ max \{ ( kz + {u}_{min} ) , {u}_{diastolic limit} \} , during diastole .\end{matrix} \qquad ( 8 )$$

Controller awareness of the cardiac cycle can be achieved using well established methods by looking at signals such as pressure (ventricular or arterial), arterial flow, electrocardiogram, echocardiography, the resulting valve body position and current, or a combination thereof.

In the case of ventricular pressure, start-systole is characterized by a sharp rise in pressure as the ventricle contracts, following a period of minimal pressure change (end diastole). Start-diastole occurs when ventricular pressure begins to fall after reaching its peak.

Start-systole and start-diastole are similarly identified using pressure in the large arteries (aorta for the left-side and pulmonary artery for the right-side). Start-systole is detected by a sudden increase in pressure, while diastole starts following the pressure peak. The dicrotic notch can be used as another indicator of start-diastole. It is a small inflection in the arterial pressure waveform caused by the closing of the arterial valve (as seen in FIG. 2 period V), detected using a first-order derivative-based algorithm to identify changes in the pressure slope.

Arterial flow measurements (aorta or pulmonary artery for left and right side, respectively) show start-systole as a rapid increase in flow when fluid is ejected from the ventricle. Start-diastole occurs when the flow rapidly decreases from the peak to zero or even a negative value (brief retrograde flow).

Electrocardiogram recording can similarly be used to identify the onset of systole and diastole. The QRS complex, representing the depolarization of the ventricles, marks the start of systole. The end of the T-wave, representing the repolarization of the ventricles, marks the start of diastole.

Imaging with echocardiography can also be used to identify start-systole and start-diastole by monitoring the state of the heart valves with real-time image detection algorithms. Start-systole occurs when the valve between the atrium and ventricle closes. Start-diastole occurs when the valve between the ventricle and large artery closes.

Using knowledge of the programmed control dynamics of the afterload, the position of the valve body can also be used to determine the onset of systole and diastole. In the simple example of spring dynamics, (6), a sudden rise in the valve body position can be used to indicate the onset of systole. Conversely, a sudden fall in the valve body position can be used to indicate the onset of diastole.

In each of the above systole-diastole detection cases where a sudden rise or fall in the signal is to be detected, standard edge detection algorithms can be employed. An example is keeping moving averages of the upper and lower envelopes of the signal of interest, then using the crossing of a threshold value between the upper and lower envelopes to detect a positive or negative edge. Another example of edge detection would be to monitor the signal derivative and set positive and negative threshold values on the first-order derivative; when the positive threshold is exceeded a positive edge has occurred, and when the negative threshold is exceeded, a negative edge has occurred. Where maximum or minimum values are to be identified, local maxima and minima detection algorithms using the first-order derivative can be employed. Alternatively, topological prominence algorithms can be used to find the local maxima and minima.

In FIG. 18 static current limits are used. However, pressure feedback can be used to update the value of the current limits, to maintain the desired pressure limits despite changes in hemodynamics. For example, a PID controller running at frequencies about 10 times faster than typical heart dynamics (i.e. 200 Hz), would be capable of rapidly adjusting the current limits to controlling pressure between the desired limits on a beat-to-beat basis.

The behavior of the afterload dynamics would be programmed to be more physiologically representative than the simple spring model, e.g. Windkessel dynamics, making use of feedback of the system state (e.g. position and/or pressure and/or flow). The simple spring dynamics would be replaced with u=ƒ(t, AP, z), where ƒ is defined by the system of equations for the desired afterload dynamics according to the previous and present state of the system.

Controlling the afterload to behave as a four-element Windkessel model as in FIG. 1, various pathological states can be simulated by setting parameter values of the four elements, or by making successive changes in the values throughout the experiment. For example, the narrowing of the aortic valve opening seen in aortic stenosis could be simulated by increasing the characteristic resistance, R_{c}. Atherosclerosis, the hardening and narrowing of the arteries, or other conditions causing hypertension, can be simulated by increasing the characteristic and peripheral resistance, R_{c}and Rₚ, as well as lowering the compliance, C. A pulmonary embolism (obstruction of the pulmonary artery) could be simulated when testing the right-side heart by increasing characteristic resistance R_{c}.

To investigate the onset of heart failure, successive changes to the Windkessel model parameters could be made until heart failure occurs. Systolic heart failure, where ejection fraction is reduced due to impaired contractile function, could be simulated by successively increasing the characteristic resistance R_{c} (representative of underlying conditions leading to hypertension) and peripheral resistance (representative of a constriction of the systemic vasculature in an attempt to compensate for the failing hearts reduced cardiac output), as well as decreasing compliance C (also representative of underlying conditions leading to hypertension), until the increased afterload on the heart leads to failure.

More generally, the model parameters could be fit to measured data using an optimization algorithm, where the measured data is representative of a particular patient, medical condition, and exertion level. The scenario captured by the data could then be represented using the afterload in an ex vivo working mode preparation by programming the afterload dynamics with the fitted Windkessel model parameters and matching other relevant parameters (cardiac output, heart rate, etc.). The same process could be followed using lower-order Windkessel models, or entirely different models.

The reference dynamics could also take on more advanced, including nonlinear, forms than the aforementioned spring or Windkessel examples. For example, a truncated transition line model, of which the Windkessel model can be viewed as a lumped-parameter approximation, could be employed. The pressure limiting dynamics could also take on a more gradual form and start altering the resistance as either of the diastolic or systolic limits are approached, rather than setting on with full effect at said limits.

Dynamic control of the afterload device relies on an estimate of its state. To this end, at least one of the following, and potentially additional, instantaneous measurements could be used: pressure at the interface of the aorta and the device; instantaneous aortic or pulmonary flow rate; flow rate entering the device from the auxiliary flow path, mechanical position (linear or angular) of the flow-obstructing moving part of the device that determines its flow impedance, energy expenditure of the actuation of said moving part (e.g. current through linear or rotary motor driving it).

A subset of these measurements could be used in isolation. They could also be used in combination both with each other and a dynamical model of the device. There exist well-established techniques to thus obtain model-based state estimation using sensor fusion, that also opens up for soft sensors to estimating internal state variables that are not directly associated with a sensor measurement, see for example “Statistical sensor fusion” by Fredrik Gustafsson, printed 2018 Mar. 7, ISBN 9789144127248 or “Handbook of Multisensor Data Fusion” edited by Martin Liggins II, David Hall, James Llinas published 2017 Jan. 31, ISBN 9781315219486.

Another approach of controlling the afterload device may be the following, with reference to the embodiment of FIG. 3. The control device 79 is adjusted to a nominal cardiac cycle period of for example 1000 ms (60 beats per minute heart rate), a nominal maximum position of the valve body of 0.75 mm, a nominal minimum position of the valve body of 0.30 mm, and a nominal auxiliary flow rate of 450 ml/min, resulting in an end-diastolic pressure of 50 mmHg. The pump 31 provides a flow rate of 5 l/min to the left atrium.

A start of a heart beat is determined, for example by monitoring a sudden rise in the current through the linear motor 93. At the start of a heart beat, the control device increases and then decreases the conductance of the controlled resistor according to a sinusoidal curve having a rise time of 15% of the cardiac cycle period, i.e. 150 ms, followed by a decrease time of 15%, i.e. 150 ms. Then, the control device keeps the conductance constant until the start of a new heart beat. Now, the control device adjusts the nominal cardiac cycle period to the actual cardiac cycle period measured and new cycle starts.

The maximal position of the valve body may be adjusted or started from a low value of for example 0.70 mm and increasing by 0.001 mm for each heart cycle until a maximum allowed systolic pressure is reached, or the reverse, starting from a high value and decreasing for each cycle.

Similarly, the minimum position of the valve body may be adjusted for each heart beat to reach a desired end-diastolic pressure.

This approach is independent of the heart pressure cycle, since a sinusoidal curve is used. However, the sinusoidal curve may be replaced by other predetermined curvatures, for example the first 15% could comprise an exponential curve, with an exponent adjusted during each cardiac cycle from a start value. Similarly, the second 15% may comprise an exponential curve decreasing by an exponent which is constant or updated.

When blood is ejected out of the ventricles, about 70% of the blood emptying occurs during the first third of the period of ejection (period of rapid ejection) and the remaining 30% emptying during the next two thirds (period of slow ejection), it may be advantageous to provide the increase of conductance during 10% of the heart beat and the decrease of conductance during 20% of the heart beat, if the systole is about 30% of the heart beat. The percent figures are given for a systolic phase of 30% of a heart beat. If the systolic phase is shorter or longer, the percent figures may be adjusted correspondingly, for example if the systolic phase is 45% of the heart beat, the increase of conductance may be 15% of the heart beat and the decrease of conductance could be 30% of the heart beat.

Additionally or alternatively, the auxiliary flow rate may be adjusted, for example lowered for each heart beat in order to result in a minimal valve position which is lower than 0.30 mm, while still keeping the desired start-diastolic pressure. The valve position may be adjusted during diastole to take into account a slow decrease of diastolic pressure during diastole.

In the claims, the term “comprises/comprising” does not exclude the presence of other elements or steps. Furthermore, although individually listed, a plurality of means, elements or method steps may be implemented by e.g. a single unit. Additionally, although individual features may be included in different claims or embodiments, these may possibly advantageously be combined, and the inclusion in different claims does not imply that a combination of features is not feasible and/or advantageous. In addition, singular references do not exclude a plurality. The terms “a”, “an”, “first”, “second” etc. do not preclude a plurality. Reference signs in the claims are provided merely as a clarifying example and shall not be construed as limiting the scope of the claims in any way.

Although the present invention has been described above with reference to specific embodiments, it is not intended to be limited to the specific form set forth herein. Rather, the invention is limited only by the accompanying claims and other embodiments than those specified above are equally possible within the scope of these appended claims.

1. A method of providing an afterload to a beating heart ex vivo, comprising
providing a first fluid flow to an atrium of the heart, whereupon the heart expels the provided fluid flow by means of a ventricle during a single heart beat in an outflow fluid flow during a systolic phase followed by a diastolic phase in which there is no outflow fluid flow from the ventricle;
passing the outflow fluid flow to an afterload device comprising an adjustable first resistor connected to the ventricle of the heart;
providing fluid flow to coronary vessels of the heart;
adjusting the first resistor to have at the start of the systolic phase a first conductance, which increases during a first part of the systolic phase and decreases during a following second part of the systolic phase during said single heart beat.

2. The method according to claim 1, further comprising
providing a second fluid flow to the connection between the afterload device and the ventricle, whereby the second fluid flow, during the diastolic phase, is divided into a third fluid flow towards the ventricle and a fourth fluid flow through the first resistor.

3. The method according to claim 2, wherein the second fluid flow is provided to the connection between the afterload device and a left ventricle of the heart, whereupon the third fluid flows towards the left ventricle during the diastolic phase, thereby closing a corresponding aortic valve and providing said third fluid flow to the coronary vessels of the heart.

4. The method according to claim 3, wherein said second flow is provided with a predetermined second fluid flow rate, which is larger than said fluid flow to the coronary vessels, and wherein the resistor is adjusted so that the fourth fluid flow through the first resistor causes a pressure drop over the first resistor, which is equal to a predetermined diastolic pressure.

5. The method according to claim 2, wherein the second fluid flow is provided by a constant pressure pump providing a constant pressure which is larger than the predetermined diastolic pressure and is provided to the connection between the afterload device and a left ventricle of the heart via a second resistor, whereby the first resistor during the diastolic phase is adjusted so that the provided constant pressure is divided to produce said predetermined diastolic pressure at said connection.

6. The method according to claim 2, wherein the second fluid flow is provided to the connection between the afterload device and a right ventricle of the heart, whereupon the third fluid flow towards the right ventricle closes a corresponding pulmonary valve, and wherein said fluid flow to coronary vessels of the heart is provided by a separated pump.

7. The method according to claim 1, further comprising:
measuring a pressure at the afterload device before the first resistor;
interrupting said decrease of the conductance during said second part of the systolic phase when said predetermined diastolic pressure has been reached.

8. The method according to claim 1, further comprising:
measuring a flow or a pressure at the afterload device before the first resistor;
interrupting the increase of the conductance when a predetermined conductance has been reached.

9. The method according to claim 7, wherein the first resistor is controlled by a motor providing a current, which is proportional to the pressure before the first resistor and which provides said measuring of the pressure before the first resistor.

10. The method according to claim 1, further comprising:
measuring a flow or a pressure at the afterload device before the first resistor and determining a start of the systolic phase when said flow or pressure exceeds a predetermined flow or pressure or increases suddenly.

11. An afterload device for providing an afterload to an outflow fluid flow expelled from a ventricle of a beating heart, comprising:
a first connector for connecting the afterload device to a ventricle of the heart for receiving the outflow fluid flow expelled by the ventricle;
an afterload body for receiving the outflow fluid flow from the ventricle;
a valve body for forming a first resistor having an adjustable first resistance and for influencing upon the outflow fluid flow through the afterload body;
a control device for receiving input data and arranged to adjust the first resistor to have at the start of a systolic phase a first conductance, which increases during a first part of the systolic phase followed by a decrease of conductance during a second part of the systolic phase;
a second connector for providing a second fluid flow to the first connection between the afterload device and the ventricle whereby the second fluid flow, during a diastolic phase is divided in a third fluid flow towards the ventricle and a fourth fluid flow through the resistor;
a pump for providing said second fluid flow;
pressure sensors and/or flow sensors for measuring pressures and/or flow rates within at least one of: atrium, ventricle, input of the afterload device, output of the afterload device, first or second fluid flows;
position meters for measuring valve body position.

12. The afterload device according to claim 11, further comprising that the afterload body is non-elastic.

13. The afterload device according to claim 11, further comprising that
the afterload body has a small volume for providing a small inertance, whereby the volume is smaller than an expelled volume of fluid by the ventricle in a cardiac beating cycle.

14. The afterload device according to claim 13, whereby the volume of the afterload body is smaller than 50 ml, such as smaller than 30 ml, for example smaller than 20 ml.

15. The afterload device according to claim 12, further comprising that
the afterload body has a small volume for providing a small inertance, whereby the volume is smaller than an expelled volume of fluid by the ventricle in a cardiac beating cycle.

16. The method according to claim 3, wherein the second fluid flow is provided by a constant pressure pump providing a constant pressure which is larger than the predetermined diastolic pressure and is provided to the connection between the afterload device and a left ventricle of the heart via a second resistor, whereby the first resistor during the diastolic phase is adjusted so that the provided constant pressure is divided to produce said predetermined diastolic pressure at said connection.

17. The method according to claim 8, wherein the first resistor is controlled by a motor providing a current, which is proportional to the pressure before the first resistor and which provides said measuring of the pressure before the first resistor.
